(19) United States
(12) Patent Application Publication
(10) Pub. No.: US 2003/0114519 A1
(43) Pub. Date: Jun. 19, 2003

(54) Pharmaceutical combination of bicalutamide and anastrozole for providing an anti-androgenic effect and aromatase inhibition

(76) Inventors: Barrington Furr, Macclesfield Cheshire (GB); Ian Cockshott, Macclesfield Cheshire (GB)

Correspondence Address: ROPES & GRAY, ONE INTERNATIONAL PLACE, BOSTON, MA, 02110-2624, US

(21) Appl. No.: 10/276,112

(22) PCT Filed: May 22, 2001
(86) PCT No.: PCT/SE01/01163

(51) Int. Cl. A61K 31/277

(52) U.S. Cl. 514/522

(57) ABSTRACT

The present invention relates to a pharmaceutical product, daily dose or dose regimen comprising 4′-cyano-α′,α′,α′-trifluoro-3-(4-fluorophenylsulphonyl)-2-hydroxy-2-methylpropriono-m-toluidide (compound I) and anastrozole. The invention also relates to a method of providing an anti-anrogenic effect and aromatase inhibition in a patient, wherein the aromatase inhibition is provided substantially without causing an additional increase in the levels of circulating androgens.

The present invention relates to a pharmaceutical product, daily dose or dose regimen comprising 4′-cyano-α′,α′,α′-trifluoro-3-(4-fluorophenylsulphonyl)-2-hydroxy-2-methylpropiono-m-toluidide and anastrozole. The invention also relates to a method of providing an anti-androgenic effect and aromatase inhibition in a patient, wherein the aromatase inhibition is provided substantially without causing an additional increase in the levels of circulating androgens. Furthermore, the invention relates to the use of 4′-cyano-α′, α′,α′-trifluoro-3-(4-fluorophenylsulphonyl)-2-hydroxy-2-methylpropiono-m-toluidide and anastrozole in the manufacture of a pharmaceutical product for this purpose.

### BACKGROUND TO THE INVENTION

Bicalutamide, a non-steroidal anti-androgen, is the racemate of 4′-cyano-α′,α′, α′-trifluoro-3-(4-fluorophenylsulphonyl)-2-hydroxy-2-methylpropiono-m-toluidide and is known by the AstraZeneca trade name CASODEX™. EP-100172 discloses 4′-cyano-α′, α′,α′-trifluoro-3-(4-fluorophenylsulphonyl)-2-hydroxy-2-methylpropiono-m-toluidide (named in EP-100172 as 4-cyano-3-trifluoromethyl-N-(3-p-fluorophenylsulphonyl-2-hydroxy-2-methylpropionyl)aniline) as the 8^{th} compound listed in the table in Example 6. The corresponding structure is shown in formula I:

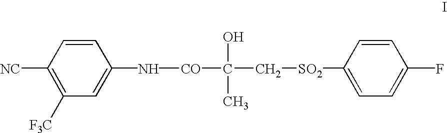

4′-cyano-α′,α′,α′-trifluoro-3-(4-fluorophenylsulphonyl)-2-hydroxy-2-methylpropiono-m-toluidide can exist in distinct R- and S-enantiomeric forms. The R-enantiomer is the (−) isomer and is the pharmacologically active compound in vivo. For further details of the enantiomers, reference is made to Tucker and Chesterton, J. Med. Chem. 31, pp 885-887 (1988). EP-100172 provides a disclosure (without supporting examples) of a pharmaceutical composition comprising 4-cyano-3-trifluoromethyl-N-(3-p-fluorophenylsulphonyl-2-hydroxy-2-methylpropionyl)aniline in combination with “one or more drugs selected from anti-oestrogens, for example tamoxifen; aromatase inhibitors, for example testolactone or aminoglutethamide; progestins, for example medroxyprogesterone acetate; inhibitors of gonadotrophin secretion, for example danazol; LH-RH-analogues, for example buserelin; cytotoxic agents, for example cyclophosphamide; antibiotics, for example penicillin or oxytetracyclin; and anti-inflammatory agents, for example, especially for topical use, fluocinolone acetonide”.

Anastrozole, an aromatase inhibitor, is known by the AstraZeneca trade name ARIMIDEX™. Anastrozole is known as 2,2′-[5-(1H-1,2,4-triazol-1-ylmethyl)-1,3-phenylene]di(2-methyl-propionitrile), which is disclosed in US re-issue No. 36,617. An alternative name is 2,2′-dimethyl-2,2′-[5-(1H-1,2,4-triazol-1-ylmethyl)-1,3-phenylene]bis(propiononitrile). The corresponding structure is shown in formula II:

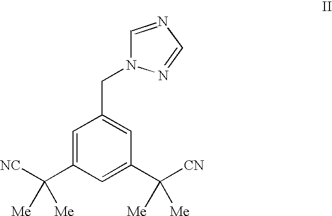

Bicalutamide can be used for the treatment of prostate cancer in combination with an inhibitor of gonadotrophin secretion, for example a luteinising hormone releasing hormone (LHRH) agonist such as goserelin, buserelin, leuprorelin or triptorelin. The properties and usefulness of bicalutamide as an anti-androgen have been reviewed in B J A Furr et al., Urology, 1996, 47 (Suppl. 1A), 13-25, and G J C Kolvenbag et al., Urology, 1996, 47 (Suppl. 1A), 70-79.

It has been observed that the administration of bicalutamide in single agent therapy to humans causes an increase in the amount of testosterone circulating in the blood. Blackledge et al, (Urology, 1996, 47, Suppl. 1A), pp 44-47) discloses an approximate doubling of the basal level of total testosterone. It is believed that such an increase in the level of testosterone occurs when sufficient of the anti-androgen gains access to the CNS and blocks androgen receptors in the hypothalamus. The consequential lack of feedback of androgen causes additional release of LHRH by the hypothalamus which in turn causes release of luteinising hormone (LH) and follicle stimulating hormone (FSH) by the pituitary gland and production of testosterone in the testes. Aromatase enzyme in fat and other tissues converts some of the increased concentration of testosterone to oestradiol, which results in increased concentrations of oestrogen in the blood. Further discussion of this is provided by C Mahler et al, Clinical Pharmacokinetics, 1998, 34(5), pp 405-417.

A disadvantageous effect is produced. Namely, the increase in the levels of circulating oestrogen may cause one or more of the side effects of gynaecomastia, breast tenderness, hot flushes, impotence and reduction in libido. A discussion on gynaecomastia can be found in C J Tyrrell, Prostate Cancer and Prostatic Diseases, 1999, 2(4): pp 167-171.

As explained above, the testosterone and LH levels tend to rise. Mahler et al explain that the rising oestrogen levels progressively activate the normal feedback mechanism, and so the rise in LH and testosterone is limited. It is widely accepted in the art that oestrogen levels are important in regulating LH secretion, and by this means testosterone secretion, as invoked by Mahler et al. It is clear from numerous publications that the reduction of the negative feedback effect of oestrogens on the hypothalamic-pituitary axis in men and male animals results in an increase in luteinising hormone (LH) secretion. This in turn drives the testes to produce increased quantities of testosterone. In this respect, reference is made to F H Comhaire et al, Human Reproduction, 1995, 10 (7), pp 1740-1744, where tamoxifen (an anti-oestrogen) intake in adult men was reported to increase testosterone and LH.

J J Spijkstra et al, J. Clinical Endocrinology and Metabolism, 1988, 66(2), pp 355-360, reports a study of LH secretion in 13 normal men before and after the administration of tamoxifen for a 6 week period. An increase in mean serum testosterone, oestradiol, LH levels, LH pulse frequency and LH pulse amplitude were observed after tamoxifen administration. Similar results were cited in men given the anti-oestrogen clomiphene citrate. Spijkstra et al suggest that the observed result with tamoxifen was due to an inhibition of negative feedback on pituitary oestrogen receptors.

D I Lewis-Jones et al, Andrologia 1987, 19(1): pp 86-90 reports that tamoxifen administration to men elevates the basal serum levels of LH, oestradiol “and particularly testosterone. The marked elevation in serum testosterone levels produced by the administration of tamoxifen may be a more successful method for elevating male hormone levels than the use of other pharmacological agents such as mesterolone”.

L van Bergeijk et al, Horm. Metabol. Res., 1986, pp 558-564, reports that three months' treatment with tamoxifen in normogonadotrophic oligozoospermic men stimulated basal LH, FSH and testosterone levels. They suggested that oestrogens play a role in the negative feedback regulation of gonadotrophin release.

There is, therefore, comprehensive evidence that would lead the skilled person in the art to reject the idea of using an anti-oestrogen to combat the rise in oestrogen levels and associated side effects observed when an anti-androgen is administered to a male. This is because the anti-oestrogen would be expected, in view of the numerous previously reported studies, to interfere with the negative feedback effect of oestrogen at the hypothalamic-pituitary axis and thus produce a substantial additional increase in LH and testosterone, which in turn would be expected to compromise the anti-androgenic effect of the anti-androgen. Furthermore, it follows from this that the skilled person would believe that the interference of this negative feedback by the use of an aromatase inhibitor would also produce a substantial additional increase in LH and testosterone, which in turn would be expected to compromise the anti-androgenic effect of the anti-androgen. There is therefore also a prejudice in the art against using an aromatase inhibitor to combat the rise in oestrogen levels and associated side effects observed when an anti-androgen is administered to a male. In addition, the skilled person would also predict that there would be an increase in testosterone due to the inhibition of its conversion to oestrogens, which would also lead the skilled person to reject the use of an aromatase inhibitor.

There is therefore a need for a treatment that can provide an anti-androgenic effect and combat the rise in oestrogen levels, thereby suppressing a side effect selected from gynaecomastia, breast tenderness, hot flushes, impotence and reduction in libido, without substantially causing an additional increase in the levels of circulating androgens above the levels produced by the anti-androgen alone.

### SUMMARY OF THE INVENTION

The present invention fulfils this need by providing a pharmaceutical product for administration to a patient for providing an anti-androgenic effect and aromatase inhibition in the patient, the product comprising a compound of formula I or a pharmaceutically acceptable salt or solvate thereof and anastrozole or a pharmaceutically acceptable salt or solvate thereof. Preferably, the compound of formula I and anastrozole are provided in a ratio of 25 to 350:0.005 to 100 respectively.

As a result of the present invention, the aromatase inhibition is provided substantially without causing an additional increase in the levels of circulating androgens. By this, we mean that the androgen levels (eg, as indicated by total or free testosterone in blood) in the patient do not substantially increase above the level usually observed when the anti-androgen alone is administered to patients.

The present invention also provides a daily pharmaceutical dose for administration to a patient for providing an anti-androgenic effect and aromatase inhibition in the patient, the dose comprising a compound of formula I or a pharmaceutically acceptable salt or solvate thereof and from 0.005 to 100 mg of anastrozole or a pharmaceutically acceptable salt or solvate thereof.

In addition, the present invention provides a dose regimen for such purpose comprising a compound of formula I or a pharmaceutically acceptable salt or solvate thereof (eg, 150 mg thereof) and from 0.005 to 100 mg of anastrozole or a pharmaceutically acceptable salt or solvate thereof for simultaneous or sequential administration to the patient.

Other aspects of the invention relate to the use in the manufacture of a pharmaceutical product of a compound of formula I or a pharmaceutically acceptable salt or solvate thereof and anastrozole or a pharmaceutically acceptable salt or solvate thereof that are simultaneously or sequentially administrable to a patient, for:

(a) providing an anti-androgenic effect and aromatase inhibition in the patient, wherein the aromatase inhibition is provided substantially without causing an additional increase in the levels of circulating androgens; or

(b) providing an anti-androgenic effect in the patient and suppressing increase in the incidence or severity of a side effect selected from gynaecomastia, breast tenderness, hot flushes, impotence and reduction in libido, substantially without causing an additional increase in the levels of circulating androgens.

By “suppressing increase in the incidence or severity of a side effect”, we mean providing a lower incidence or severity compared with the side effect produced when the anti-androgen is administered alone, or eliminating the side effect.

The present invention further provides a method of providing an anti-androgenic effect in a patient comprising simultaneously or sequentially administering a compound of formula I or a pharmaceutically acceptable salt or solvate thereof and anastrozole or a pharmaceutically acceptable salt or solvate thereof to the patient, wherein the method further provides aromatase inhibition in the patient substantially without causing an additional increase in the levels of circulating androgens.

### DETAILED DESCRIPTION OF THE INVENTION

We have now surprisingly found that both an anti-androgenic effect and aromatase inhibition can be produced in a patient, wherein the aromatase inhibition is provided substantially without causing an additional increase in the levels of circulating androgens. This is achieved by administering to the patient a product comprising a compound of formula I or a pharmaceutically acceptable salt or solvate thereof and anastrozole or a pharmaceutically acceptable salt or solvate thereof. Preferably, the compound of formula I and anastrozole are provided in a ratio respectively of 25 to 350 (preferably the lower end of the range being 50; preferably the upper end of the range being 300, 150 or 50; suitable values in the ranges being 150 or 50): 0.005 to 100 (preferably the lower end of the range being 0.05 or 0.5; preferably the upper end of the range being 50, 10 or 1; the most preferred range being 0.5 to 1; a suitable value in the range being 1). The term “product” is intended to mean either a mixture of the compound of formula I and anastrozole (eg, provided as a capsule or tablet containing both compounds) or a kit comprising separate amounts of the compounds (eg, a set of anastrozole tablets and a separate set of tablets of the other compound). The latter product can be used for simultaneous or sequential (ie, temporally spaced) administration of the compounds to the patient, while the pre-mixed compounds are for simultaneous administration. Factors such as the rate of absorption, metabolism and the rate of excretion of each agent will affect their presence at the tumour site. Such factors are routinely considered by, and are well within the ordinary skill of, the clinician when he contemplates the treatment of a medical condition which requires the conjoint administration of two agents in order to obtain a beneficial effect.

The compound of formula I is included to provide an anti-androgenic effect, in that this compound blocks androgen activity. The anastrozole is included to provide aromatase inhibition, in that this compound inhibits conversion of testosterone to oestradiol by aromatase enzyme.

The anti-androgenic effect is useful for treating cancer, for example prostate cancer. Particular examples are advanced prostate cancer and early prostate cancer. The anti-androgenic effect may be useful for prophylaxis, in order to reduce the risk of prostate cancer occurrence in patients. This could be especially useful in men genetically predisposed to prostate cancer. Conventional methods are available to classify patients according to their risk of contracting prostate cancer, for example by assessment of family history and measurements over time of particular blood proteins such as prostate specific antigen (PSA). Other uses for the anti-androgenic effect are the treatment of a non-malignant disease of the prostate gland (eg, benign prostatic hyperplasia or hypertrophy) and acne.

The aromatase inhibition is useful for suppressing increase in the incidence or severity of a side effect selected from gynaecomastia, breast tenderness, hot flushes, impotence, reduction in libido, nausea, vomiting, fatigue and diarrhoea. Such side effects have been observed with monotherapy use of anti-androgens. Preferably, the side effect is one or both of gynaecomastia and breast tenderness.

A suitable dose regimen or daily pharmaceutical dose comprises the compound of formula I or a pharmaceutically acceptable salt or solvate thereof and from 0.005 to 100 mg of anastrozole or a pharmaceutically acceptable salt or solvate thereof. Preferably, for the amount of anastrozole the lower end of the range is 0.05 or 0.5 mg; preferably the upper end of the range is 50, 10 or 1 mg; the most preferred range is 0.5 to 1 mg; a suitable value in the range being 1 mg. The dose or the regimen preferably comprises from 25 to 350 mg of the compound of formula I or a pharmaceutically acceptable salt or solvate thereof. Preferably the lower end of the range is 50 mg; preferably the upper end of the range is 300, 150 or 50 mg; suitable values in the ranges are 150 or 50 mg.

For the regimen, each compound is preferably administered daily. Another possible regime would be dosing of the compound of formula I on alternate days and dosing of the anastrozole also on (the same or different) alternate days. To this end, the regimen may include administration instructions. Preferably, a dose of the compound of formula I is administered every 3, 4, 5, 6 or 7 days and the anastrozole is administered every 3, 4, 5, 6 or 7 days (eg, on the same day as the compound of formula I).

In one embodiment of the present invention, the compound of formula I consists of 90 to 100% of the R-enantiomer and 10 to 0% of the S-enantiomer thereof. In a preferred embodiment, 100% of the R-enantiomer is used.

In another embodiment, the compound of formula I consists of a racemic mixture of the R- and S-enantiomers thereof.

The patient can be a human male, eg an adult, but the treatment of other mammals (except rats) is also contemplated.

The products, doses and regimens of the invention may be in a form suitable for oral use (for example as tablets, capsules, aqueous or oily suspensions, emulsions or dispersible powders or granules), for topical use (for example as creams, ointments, gels, or aqueous or oily solutions or suspensions; for example for use within a transdermal patch), for parenteral administration (for example as a sterile aqueous or oily solution or suspension for intravenous, subcutaneous, intramuscular or intravascular dosing), or as a suppository for rectal dosing. Preferably the compositions of the invention are in a form suitable for oral use, for example as tablets or capsules.

The products, doses and regimens of the invention may be obtained by conventional procedures using conventional pharmaceutically-acceptable diluents or carriers that are well known in the art.

Suitable pharmaceutically-acceptable diluents or carriers for a tablet formulation include, for example, inert diluents such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin or olive oil.

When we mention providing aromatase inhibition without causing an additional increase in the levels of circulating androgens, we mean that the androgen levels (eg, as indicated by total or free testosterone in blood) in the patient do not substantially increase above the maximum level usually observed when the anti-androgen alone is administered to patients. An enabling illustration is provided in the human clinical trial below.

Human Clinical Trial

The following clinical trial was performed to determine the effect of the administration of CASODEX™ together with ARIMIDEX™ on free testosterone levels in healthy male volunteers over a 6 week period.

Protocol

Key Inclusion Criteria: Male, aged 65 years or above showing no clinically significant abnormalities in routine haematological and biochemical tests and having endocrinology and prostate specific antigen (PSA) results within normal limits.

Key Exclusion Criteria: Previous inclusion in a clinical trial using CASODEX™; concurrent treatment with any drugs with the exception of paracetomol; history or presence of any testicular abnormality; history or presence of gastrointestinal, hepatic or renal disease, or other condition known to interfere with the absorption, distribution, metabolism or excretion of drugs; a clinically significant illness within 2 weeks of trial commencement; definite or suspected personal or family history of significant adverse drug reactions or any hypersensitivity to CASODEX™ or ARIMIDEX™; treatment within the previous 3 months with any drugs known to have a well-defined potential for hepatotoxicity or hepatic interaction.

Dosage: The CASODEX™ was administered daily at a dose of 150 mg and the ARIMIDEX™ was administered daily at a dose of 1 mg. All treatments were in tablet form and taken once daily. Daily treatment with CASODEX™ was for 6 weeks, and with ARIMIDEX™ for the final 2 weeks of this period. The treatment periods were selected as the minimum time to attain steady-state plasma concentrations for the drugs.

Key Assessment: Free testosterone concentrations were measured during the course of the trial.

Results

A summary of the free testosterone concentrations over the treatment periods is presented in Table 1.

TABLE 1
Free testosterone concentrations following
treatment with CASODEX ™ alone
(up to week 4) plus ARIMIDEX ™ (after week 4)

| | | | | | |
|---|---|---|---|---|---|
| Parameter | Day 1 | Day 29 | Day 36 | Day 43 | Follow-up |

Test-
osterone
(nmol/1)

| | | | | | |
|---|---|---|---|---|---|
| n | 7 | 7 | 7 | 7 | 7 |
| gmean | 0.048 | 0.076 | 0.075 | 0.074 | 0.049 |
| CV | 30.415 | 26.219 | 45.199 | 46.883 | 36.081 |
| Minimum | 0.03-0.07 | 0.00-0.12 | 0.03-0.11 | 0.03-0.13 | 0.03-0.09 |
| Ratio to | — | 1.58 | 1.56 | 1.54 | 1.01 |
| Day 1 | | | | | |

CV = Coefficient of variation gmean = Geometric mean n = Number of observations Day 1 samples were drawn before dosing, and therefore act as a baseline measurement. No volunteers experienced gynaecomastia.

Conclusion

When CASODEX™ alone was administered, the mean free testosterone concentration increased 58% by the end of the treatment period. With continued administration of CASODEX™ beyond the 4^{th} week, this figure would be expected to rise (corresponding to an approximate doubling of the mean total testosterone concentration). In this respect, reference is made to a trial reported by Verhelst, J et al (“Endocrine profiles during administration of the new non-steroidal anti-androgen Casodex in prostate cancer”, Verheist, J et al, Clin. Endocrinol. (Oxf) 1994, Oct., 41(4), pp 525-30), which reported an increase of 57% in the mean free testosterone concentration after 24 weeks of daily administration of 150 mg CASODEX™ alone.

Reference to Table 1 shows that the co-administration of ARIMIDEX™ with CASODEX™ produced no additional clinically significant change in the mean concentration of free testosterone. By the end of the treatment period the increase in the mean concentration was 54%.

The results therefore support our surprising finding that in the present invention the anastrozole does not compromise the anti-androgenic effect of the anti-androgen of formula I, in that contrary to the expectations of the skilled person based on the aforementioned prejudice in the art, the use of the anastrozole does not cause an additional increase in the levels of androgens beyond the levels expected when anti-androgen alone is used.

1. A pharmaceutical product for administration to a patient for providing an anti-androgenic effect and aromatase inhibition in the patient, the product comprising a compound of formula I or a pharmaceutically acceptable salt or solvate thereof and anastrozole or a pharmaceutically acceptable salt or solvate thereof.

2. The pharmaceutical product of claim 1, wherein the compound of formula I and anastrozole are provided in a ratio of 25 to 350:0.005 to 100 respectively.

3. A daily pharmaceutical dose for administration to a patient for providing an anti-androgenic effect and aromatase inhibition in the patient, the dose comprising a compound of formula I or a pharmaceutically acceptable salt or solvate thereof and from 0.005 to 100 mg of anastrozole or a pharmaceutically acceptable salt or solvate thereof.

4. A dose regimen for providing an anti-androgenic effect and aromatase inhibition in a patient, the regimen comprising a compound of formula I or a pharmaceutically acceptable salt or solvate thereof and from 0.005 to 100 mg of anastrozole or a pharmaceutically acceptable salt or solvate thereof for simultaneous or sequential administration to the patient.

5. The dose of claim 3, or the regimen of claim 4, comprising from 25 to 350 mg of the compound of formula I or a pharmaceutically acceptable salt or solvate thereof.

6. A dose regimen for providing an anti-androgenic effect and aromatase inhibition in a patient, the regimen comprising 150 mg a compound of formula I or a pharmaceutically acceptable salt or solvate thereof and from 0.005 to 100 mg of anastrozole or a pharmaceutically acceptable salt or solvate thereof for simultaneous or sequential administration to the patient.

7. Use in the manufacture of a pharmaceutical product of a compound of formula I or a pharmaceutically acceptable salt or solvate thereof and anastrozole or a pharmaceutically acceptable salt or solvate thereof for simultaneous or sequential administration to a patient, for providing an anti-androgenic effect and aromatase inhibition in the patient, wherein the aromatase inhibition is provided substantially without causing an additional increase in the levels of circulating androgens.

8. Use in the manufacture of a pharmaceutical product of a compound of formula I or a pharmaceutically acceptable salt or solvate thereof and anastrozole or a pharmaceutically acceptable salt or solvate thereof for simultaneous or sequential administration to a patient, for providing an anti-androgenic effect in the patient and suppressing increase in the incidence or severity of at least one side effect selected from gynaecomastia, breast tenderness, hot flushes, impotence and reduction in libido, substantially without causing an additional increase in the levels of circulating androgens.

9. A method of providing an anti-androgenic effect in a patient comprising simultaneously or sequentially administering a compound of formula I or a pharmaceutically acceptable salt or solvate thereof and anastrozole or a pharmaceutically acceptable salt or solvate thereof to the patient, wherein the method further provides aromatase inhibition in the patient substantially without causing an additional increase in the levels of circulating androgens.

10. The product, dose, regimen, use or method of any preceding claim, wherein the compound of formula I consists of 90 to 100% of the R-enantiomer and 10 to 0% of the S-enantiomer thereof.

11. The product, dose, regimen, use or method of any of claims 1 to 9, wherein the compound of formula I consists of a racemic mixture of the R- and S-enantiomers thereof.
